(19) United States
(12) Patent Application Publication
(10) Pub. No.: US 2020/0129421 A1
(43) Pub. Date: Apr. 30, 2020

(54) DENTAL TRAY AND USE THEREOF

(71) Applicant: Hui Feng, Shanghai (CN)

(72) Inventor: Hui Feng, Shanghai (CN)

(21) Appl. No.: 16/663,296

(22) Filed: Oct. 24, 2019

Related U.S. Application Data
(60) Provisional application No. 62/749,903, filed on Oct. 24, 2018.

(51) Int. Cl. A61K 9/00; A61K 8/02; A61K 9/70; A61Q 11/00

(52) U.S. Cl. CPC A61K 9/006; A61K 8/0204; A61K 2800/56; A61K 9/703; A61Q 11/00; A61K 9/0002

(57) ABSTRACT

Provided herein is a system for delivery of a physiologically active agent to a user in need thereof, comprising: one or two appliances that correspond to an upper arch and/or lower dental arch of the user, each appliance comprising a labial/buccal side surface and a palatal/lingual side surface, a delivery module of a physiologically active agent, comprising the physiologically active agent, the one or two appliances comprise a body matrix of a polymeric material comprising polymeric shells, each shell having the geometry corresponding to one tooth of the dental arch(s) and configured to receive the one tooth, and the delivery module provides a controlled release of the physiologically active agent.

### CROSS-REFERENCE TO RELATED APPLICATIONS

This application claims the benefit of U.S. provisional application Ser. No. 62/749,903, filed Oct. 24, 2018, the teaching of which being incorporated herein by reference in its entirety.

### FIELD OF THE INVENTION

The present invention relates generally to the field of dental technology.

### BACKGROUND OF THE INVENTION

Conventional method of repositioning teeth is to make users wear what are commonly referred to as “braces.” Braces include a variety of appliances such as brackets, archwires, ligatures, and O-rings. The procedures of conventional orthodontics are briefly described here: Before fastening braces to a user's teeth, at least one appointment is typically scheduled with the orthodontist, dentist, and/or X-ray laboratory so that X-rays and photographs of the user's teeth and jaw structure can be taken. Also during this preliminary meeting, or possibly at a later meeting, an alginate mold of the user's teeth is typically made. This mold provides a model of the user's teeth that the orthodontist uses in conjunction with the X-rays and photographs to formulate a treatment strategy. The orthodontist then typically schedules one or more appointments during which braces will be attached to the user's teeth.

At the meeting during which braces are first attached, the teeth surfaces are initially treated with a weak acid the acid optimizes the adhesion properties of the teeth surfaces for brackets and bands that are to be bonded to them. The brackets and bands serve as anchors for other appliances to be added later. After the acid step, the brackets and bands are cemented to the user's teeth using a suitable bonding material. No force-inducing appliances are added until the cement is set. For this reason, it is common for the orthodontist to schedule a later appointment to ensure that the brackets and bands are well bonded to the teeth.

The primary force-inducing appliance in a conventional set of braces is the archwire. The archwire is resilient and is attached to the brackets by way of slots in the brackets. The archwire links the brackets together and exerts forces on them to move the teeth over time. Twisted wires or elastomeric O-rings are commonly used to reinforce attachment of the archwire to the brackets. Attachment of the archwire to the brackets is known in the art of orthodontia as “ligation” and wires used in this procedure are called “ligatures.” The elastomeric O-rings are called “plastics.”

After the archwire is in place, periodic meetings with the orthodontist are required, during which the user's braces will be adjusted by installing a different archwire having different force inducing properties or by replacing or tightening existing ligatures. Typically, these meetings are scheduled every three to six weeks.

Therefore, attaching the appliances to a user's teeth is tedious and time-consuming enterprise and requires many meetings with the treating orthodontist.

Various methods have been developed to simplify and to reduce the cost of orthodontics with limited success. For example, Kuroda et al. (1996) Am. J. Orthodontics 110:365-369 describes a method for laser scanning a plaster dental cast to produce a digital image of the cast. See also U.S. Pat. No. 5,605,459. U.S. Pat. Nos. 5,533,895; 5,474,448; 5,454,717; 5,447,432; 5,431,562; 5,395,238; 5,368,478; and 5,139,419, assigned to Ormco Corporation, describe methods for manipulating digital images of teeth for designing orthodontic appliances.

FIGS. 1A-1C show clear aligners. FIGS. 1A and 1B show clear aligners of a lower dental arch, having a labial/buccal side and a palatal/lingual side. FIG. 1C show an aligner of a upper dental arch, having a labial/buccal side and a palatal/lingual side.

U.S. Pat. No. 5,011,405 describes a method for digitally imaging a tooth and determining optimum bracket positioning for orthodontic treatment. Laser scanning of a molded tooth to produce a three-dimensional model is described in U.S. Pat. No. 5,338,198. U.S. Pat. No. 5,452,219 describes a method for laser scanning a tooth model and milling a tooth mold. Digital computer manipulation of tooth contours is described in U.S. Pat. Nos. 5,607,305 and 5,587,912. Computerized digital imaging of the jaw is described in U.S. Pat. Nos. 5,342,202 and 5,340,309. Other patents of interest include U.S. Pat. Nos. 5,549,476; 5,382,164; 5,273,429; 4,936,862; 3,860,803; 3,660,900; 5,645,421; 5,055,039; 4,798,534; 4,856,991; 5,035,613; 5,059,118; 5,186,623; and 4,755,139.

U.S. Pat. Nos. 6,398,548 and 6,554,611 describes using a computer program predict the teeth movement from initial tooth positions to the final tooth positions and design and fabricate a series of appliances having different geometries to achieve the repositioning of the teeth from their initial positions to their final positions in one set of appliances in that the user receives a whole set of appliances from the beginning to the finish. The appliances are numbered or marked in the sequence for use. The problem is that if certain tooth movement is behind the expected tooth position, the discrepancy between actual tooth position and expected (predicted) tooth position will most likely increase further and further during the course of repositioning. In addition, teeth with increased discrepancies between actual and expected positions will experience a disproportionate load of force relative to the other teeth and lead to increased patient pain and discomfort.

While tooth-realigning using tooth aligning appliances are common, there is no technology on using tooth aligning appliance as a drug delivery platform. Additionally, a large part of the reason underpinning orthodontic treatment is of aesthetic nature, which includes the use of clear aligners. However, the existing technology fails to recognize and address the many un-met needs in this regard, e.g., tooth discoloration and beyond.

The embodiments described below address the above identified needs and issues.

### SUMMARY OF THE SYSTEM

In an aspect of the present invention, it is provided a system for delivery of a physiologically active agent to a user in need thereof, which system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention system disclosed herein, the delivery module further comprises a carrier material, and the physiologically active agent is admixed with, embedded within or encapsulated within the carrier material.

In some embodiments, optionally in combination with any one of the embodiments of invention system disclosed herein, the delivery module is embedded within the body matrix of the polymeric material or is admixed with the polymeric material forming the one or two appliances.

In some embodiments, optionally in combination with any one of the embodiments of invention system disclosed herein, the delivery module is formed of the physiologically active agent per se.

In some embodiments, optionally in combination with any one of the embodiments of invention system disclosed herein, the delivery module is a coating on a surface of the one or two appliances.

In some embodiments, optionally in combination with any one of the embodiments of invention system disclosed herein, the one or two appliances comprises a structure selected from the group consisting of micropores, microchannels, depots, and a combination thereof.

In some embodiments, optionally in combination with any one of the embodiments of invention system disclosed herein, the structure comprises discrete or interconnecting micropores, microchannels, depots, or a combination thereof.

In some embodiments, optionally in combination with any one of the embodiments of invention system disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention system disclosed herein, the physiologically active agent is insulin, a fluoride, or an antibiotic agent.

In some embodiments, optionally in combination with any one of the embodiments of invention system disclosed herein, the system further comprises a second agent, wherein the second agent is flavoring agent or a fragrance.

In some embodiments, optionally in combination with any one of the embodiments of invention system disclosed herein, the system further comprises a top layer of coating on a surface of the one or two appliances, wherein the top layer comprises the second agent and an optional carrier material.

In some embodiments, optionally in combination with any one of the embodiments of invention system disclosed herein, the second agent is a female or male sex hormone.

In some embodiments, optionally in combination with any one of the embodiments of invention system disclosed herein, the second agent is a flavoring agent selected from a taste of chocolate, vanilla, mint, a fruit, or candy.

In some embodiments, optionally in combination with any one of the embodiments of invention system disclosed herein, the second agent is a fragrance capable of masking a bad breath or mouse odor.

In some embodiments, optionally in combination with any one of the embodiments of invention system disclosed herein, the one or more appliances further comprise a decorative surface.

In some embodiments, optionally in combination with any one of the embodiments of invention system disclosed herein, the one or more appliances are orthodontic aligning appliances.

In some embodiments, optionally in combination with any one of the embodiments of invention system disclosed herein, the occlusal bottoms of at least one or more of the polymeric shells are removed such that the occlusal surfaces of at least one or more of the polymeric shells are exposed.

In another aspect of the present invention, it is provided a method for fabricating a system for delivery of a physiologically active agent to a user in need thereof, which method comprising:

providing one or two appliances that correspond to an upper arch and/or lower dental arch of the user, each appliance comprising a labial/buccal side surface and a palatal/lingual side surface,

providing a delivery module of a physiologically active agent, comprising the physiologically active agent, and

forming the system,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the system which system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the delivery module further comprises a carrier material, and the physiologically active agent is admixed with, embedded within or encapsulated within the carrier material.

In some embodiments, optionally in combination with any one of the embodiments of invention method disclosed herein, the delivery module is embedded within the body matrix of the polymeric material or is admixed with the polymeric material forming the one or two appliances.

In some embodiments, optionally in combination with any one of the embodiments of invention method disclosed herein, the delivery module is formed of the physiologically active agent per se.

In some embodiments, optionally in combination with any one of the embodiments of invention method disclosed herein, the delivery module is a coating on a surface of the one or two appliances.

In some embodiments, optionally in combination with any one of the embodiments of invention method disclosed herein, the one or two appliances comprises a structure selected from the group consisting of micropores, microchannels, depots, and a combination thereof.

In some embodiments, optionally in combination with any one of the embodiments of invention method disclosed herein, the structure comprises discrete or interconnecting micropores, microchannels, depots, or a combination thereof.

In some embodiments, optionally in combination with any one of the embodiments of invention method disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention method disclosed herein, the physiologically active agent is insulin, a fluoride, or an antibiotic agent.

In some embodiments, optionally in combination with any one of the embodiments of invention method disclosed herein, the system further comprises a second agent, wherein the second agent is flavoring agent or a fragrance.

In some embodiments, optionally in combination with any one of the embodiments of invention method disclosed herein, the system further comprises a top layer of coating on a surface of the one or two appliances, wherein the top layer comprises the second agent and an optional carrier material.

In some embodiments, optionally in combination with any one of the embodiments of invention method disclosed herein, the second agent is a female or male sex hormone.

In some embodiments, optionally in combination with any one of the embodiments of invention method disclosed herein, the second agent is a flavoring agent selected from a taste of chocolate, vanilla, mint, a fruit, or candy.

In some embodiments, optionally in combination with any one of the embodiments of invention method disclosed herein, the second agent is a fragrance capable of masking a bad breath or mouse odor.

In some embodiments, optionally in combination with any one of the embodiments of invention method disclosed herein, the one or more appliances further comprise a decorative surface.

In some embodiments, optionally in combination with any one of the embodiments of invention method disclosed herein, the one or more appliances are orthodontic aligning appliances.

In some embodiments, optionally in combination with any one of the embodiments of invention method disclosed herein, the occlusal bottoms of at least one or more of the polymeric shells are removed such that the occlusal surfaces of at least one or more of the polymeric shells are exposed.

In a further aspect of the present invention, it is provided a method of treating or ameliorating a condition in a user, comprising delivering to a user in need thereof a physiologically active agent to the user using a system, the system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the delivery module further comprises a carrier material, and the physiologically active agent is admixed with, embedded within or encapsulated within the carrier material.

In some embodiments, optionally in combination with any one of the embodiments of invention method disclosed herein, the delivery module is embedded within the body matrix of the polymeric material or is admixed with the polymeric material forming the one or two appliances.

In some embodiments, optionally in combination with any one of the embodiments of invention method disclosed herein, the delivery module is formed of the physiologically active agent per se.

In some embodiments, optionally in combination with any one of the embodiments of invention method disclosed herein, the delivery module is a coating on a surface of the one or two appliances.

In some embodiments, optionally in combination with any one of the embodiments of invention method disclosed herein, the one or two appliances comprises a structure selected from the group consisting of micropores, microchannels, depots, and a combination thereof.

In some embodiments, optionally in combination with any one of the embodiments of invention method disclosed herein, the structure comprises discrete or interconnecting micropores, microchannels, depots, or a combination thereof.

In some embodiments, optionally in combination with any one of the embodiments of invention method disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention method disclosed herein, the physiologically active agent is insulin, a fluoride, or an antibiotic agent.

In some embodiments, optionally in combination with any one of the embodiments of invention method disclosed herein, the system further comprises a second agent, wherein the second agent is flavoring agent or a fragrance.

In some embodiments, optionally in combination with any one of the embodiments of invention method disclosed herein, the system further comprises a top layer of coating on a surface of the one or two appliances, wherein the top layer comprises the second agent and an optional carrier material.

In some embodiments, optionally in combination with any one of the embodiments of invention method disclosed herein, the second agent is a female or male sex hormone.

In some embodiments, optionally in combination with any one of the embodiments of invention method disclosed herein, the second agent is a flavoring agent selected from a taste of chocolate, vanilla, mint, a fruit, or candy.

In some embodiments, optionally in combination with any one of the embodiments of invention method disclosed herein, the second agent is a fragrance capable of masking a bad breath or mouse odor.

In some embodiments, optionally in combination with any one of the embodiments of invention method disclosed herein, the one or more appliances further comprise a decorative surface.

In some embodiments, optionally in combination with any one of the embodiments of invention method disclosed herein, the one or more appliances are orthodontic aligning appliances.

In some embodiments, optionally in combination with any one of the embodiments of invention method disclosed herein, the occlusal bottoms of at least one or more of the polymeric shells are removed such that the occlusal surfaces of at least one or more of the polymeric shells are exposed.

In some embodiments, optionally in combination with any one of the embodiments of invention method disclosed herein, the condition is a chronic condition or an acute-on-chronic condition.

In some embodiments, optionally in combination with any one of the embodiments of invention method disclosed herein, the condition is a dental condition.

In some embodiments, optionally in combination with any one of the embodiments of invention method disclosed herein, the condition is a periodontal condition.

In some embodiments, optionally in combination with any one of the embodiments of invention method disclosed herein, the condition is a diabetic condition or a neurodegernative condition.

### BRIEF DESCRIPTION OF DRAWINGS

FIGS. 1A-1C show typical plastic appliances: 1A, 1B—for the lower dental arch, and 1C—for upper dental arch, where the polymer shells each receive and enclose the entire surface of a tooth on a dental arch.

### DESCRIPTION OF DETAILED EMBODIMENTS

### Definitions

As used herein, the term “physiologically active agent” refers to a chemical or biological compound effective for bringing about a physiological response in a user of the physiologically active agent. Such physiologically active agent can be therapeutic agent or a non-therapeutic agent.

As used herein, the term “controlled release” or “controlled delivery” refers to release of a physiologically active agent in an effective amount from a system of invention into a user when the system is exposed to moisture in the oral cavity of a user of the appliance or the appliance is exposed to a fluid, e.g., saliva, of the user of appliance. Release of the physiologically active agent is controlled by the characteristics of the delivery module, which is described in more detail below.

As used herein, the term “effective amount” refers to an amount of the physiologically active agent that produces a statistically significant positive result in a physiological condition in a user in need thereof as compared to a control not using the physiologically active agent. In this context, a physiological condition refers to a condition, which is also referred to as “disorder” or “disease” from time to time and requires medical attention, or a physiological condition that requires no medical attention, such as body odor, mouth odor, or bad breath, a condition of pure physiological pleasantness or lack thereof, such as flavor, taste, or a condition of desire, e.g., sexual attraction or lack thereof.

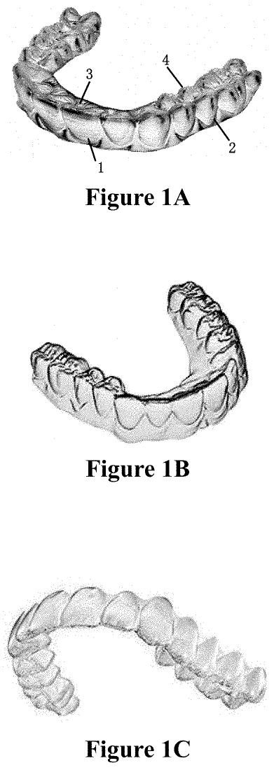

As used herein, the term “appliance” or “appliances” or “tray” or “trays” refer to a device having shells of a geometry defined by a dental arch of a user, each shell corresponding to one tooth on the dental arch and receiving only the one tooth. The appliance is formed of a plastic material and is generally clear and colorless. When an appliance or appliances are used to apply an orthodontic re-aligning or repositioning force on a tooth or teeth of a dental arch, the appliance or appliances are also called as “aligner” or “aligners”. FIGS. 1A-1C show typical plastic appliances: 1A, 1B—for the lower dental arch, and 1C—for upper dental arch, where the polymer shells each receive and enclose the entire surface of a tooth on a dental arch. Methods of forming an appliance is well documented, and examples of digital methods of forming an appliance are described in U.S. Pat. Nos. 5,605,459; 5,533,895; 5,474,448; 5,454,717; 5,447,432; 5,431,562; 5,395,238; 5,368,478; 5,139,419; 5,338,198; 5,452,219; 5,607,305; 5,587,912; 5,342,202; 5,340,309; 5,549,476; 5,382,164; 5,273,429; 4,936,862; 3,860,803; 3,660,900; 5,645,421; 5,055,039; 4,798,534; 4,856,991; 5,035,613; 5,059,118; 5,186,623; 4,755,139; 6,398,548 and 6,554,611. The teachings in each of these patents are incorporated herein in their entirety.

Whenever used, the term “duplicate” or “duplicates” or “copy” refers to appliances that are identical to each other with respect to geometries, materials forming the appliances, and/or physical dimensions of the appliances. Relatedly, the term “copy” of an appliance (“parent appliance”) refers to a copy appliance of the parent appliance that is identical to the parent appliance with respect to the geometries, materials forming the appliances, and/or physical dimensions of the appliances, and, in this context, the term “duplicate appliances” is used interchangeably with the term “duplicates” or “duplicate appliances” and refers to a copy appliance of the parent appliance that is identical to the parent appliance with respect to each of the geometries, materials forming the appliances, and physical dimensions of the appliances.

As used herein, the term “non-transparent” refers to the attribute of an appliance of invention that renders the original color of tooth/teeth of a user of the appliance invisible or substantially invisible.

As used herein, the term “translucent” refers to light being permitted to pass through but diffusing it so that persons, objects, etc., on the opposite side are not clearly visible.

### System for Controlled Delivery of Physiologically Active Agent

In an aspect of the present invention, it is provided a system for delivery of a physiologically active agent to a user in need thereof, which system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention system disclosed herein, the delivery module further comprises a carrier material, and the physiologically active agent is admixed with, embedded within or encapsulated within the carrier material.

In some embodiments, optionally in combination with any one of the embodiments of invention system disclosed herein, the delivery module is embedded within the body matrix of the polymeric material or is admixed with the polymeric material forming the one or two appliances.

In some embodiments, optionally in combination with any one of the embodiments of invention system disclosed herein, the delivery module is formed of the physiologically active agent per se.

In some embodiments, optionally in combination with any one of the embodiments of invention system disclosed herein, the delivery module is a coating on a surface of the one or two appliances.

In some embodiments, optionally in combination with any one of the embodiments of invention system disclosed herein, the one or two appliances comprises a structure selected from the group consisting of micropores, microchannels, depots, and a combination thereof.

In some embodiments, optionally in combination with any one of the embodiments of invention system disclosed herein, the structure comprises discrete or interconnecting micropores, microchannels, depots, or a combination thereof.

In some embodiments, optionally in combination with any one of the embodiments of invention system disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention system disclosed herein, the physiologically active agent is insulin, a fluoride, or an antibiotic agent.

In some embodiments, optionally in combination with any one of the embodiments of invention system disclosed herein, the system further comprises a second agent, wherein the second agent is flavoring agent or a fragrance.

In some embodiments, optionally in combination with any one of the embodiments of invention system disclosed herein, the system further comprises a top layer of coating on a surface of the one or two appliances, wherein the top layer comprises the second agent and an optional carrier material.

In some embodiments, optionally in combination with any one of the embodiments of invention system disclosed herein, the second agent is a female or male sex hormone.

In some embodiments, optionally in combination with any one of the embodiments of invention system disclosed herein, the second agent is a flavoring agent selected from a taste of chocolate, vanilla, mint, a fruit, or candy.

In some embodiments, optionally in combination with any one of the embodiments of invention system disclosed herein, the second agent is a fragrance capable of masking a bad breath or mouse odor.

In some embodiments, optionally in combination with any one of the embodiments of invention system disclosed herein, the one or more appliances further comprise a decorative surface.

In some embodiments, optionally in combination with any one of the embodiments of invention system disclosed herein, the one or more appliances are orthodontic aligning appliances.

### Delivery Module

As used herein, the term “delivery matrix” refers to a delivery module for controlled release of a physiologically active agent from a dental tray. Such delivery module useful in the present invention is an integral part of the dental tray, but is additionally added to the dental appliances or added to a material forming the dental appliances. The delivery module includes the physiologically active agent to be released to the user in need thereof.

In some embodiments, the delivery module can include the physiologically active agent per se and an optional carrier. The delivery module can be admixed with or embedded within a material forming the dental appliances.

In some embodiments, the delivery module can be loaded to pores, micropores or channels formed on the dental appliances. Such micropores, depots, or channels can be discrete or interconnected.

In some embodiments, the delivery module can be coated onto the dental appliances in the form of a coating. In some embodiments, the coating can be formed on the lingual/palatal side surface of the trays or appliances. In some embodiments, the coating can be formed on the labial/buccal side surface of the trays or appliances. In some further embodiments, the coating can be formed on inner walls of the shells of the trays or appliances.

In some further embodiments, an optional top layer can be formed on the delivery module comprising the physiologically active agent so as to control the release of the agent. The top layer can comprise a carrier material alone without a physiologically active agent or can comprise a carrier with another physiologically active agent. In this embodiment, when two physiologically active agents are included in the trays or appliances, the physiologically active agent in the delivery module can be referred to as the first agent, and the physiologically active agent in the top layer can be referred to as the second agent. Such second agent can be same as or different from the first agent in the delivery module. When the second agent is the same as the first agent, the second agent and the first agent can have different release profiles, depending upon the characteristics and configuration of the delivery module and the top layer, for example, the nature of the carrier material, e.g., hydrophobicity, forming the top layer, the thickness of the top layer, the characteristics of the delivery module, e.g., the form of the delivery module, such as the first agent being discrete particles, a mixture with a carrier material of the delivery module, such as solid solution or dispersion, or being encapsulated within the carrier material.

In some embodiments, the second agent is different from the first agent. In these embodiments, the second agent can be a flavoring agent or a fragrance.

### Physiologically Active Agent

The system described herein can be used for controlled delivery of many physiologically active agents. The invention system offers the advantages of allowing a physiologically active agent to be delivered to a subject in need thereof through the various sites of the oral cavity, e.g., lingual, sublingual, periodontal, which are established routes for effective drug delivery, by-passing the gastric track. As such, therapeutics not suitable for oral delivery, such as peptide agents or protein drugs, can be delivered to a subject using the invention system. Additionally, the system of invention offers an advantage for senile or sustained and controlled release of a physiologically active agent without having to take the physiologically active agent at a specific given time or dose, allowing medication on a physically or mentally compromised patient, e.g., patients.

The physiologically active agent can be therapeutic agent or a non-therapeutic agent. In some embodiments, from the group consisting of a protein, a peptide, a small molecule therapeutic agent, an antimicrobial agent, and a combination thereof.

In some embodiments, the physiologically active agent is insulin, a fluoride (e.g., sodium fluoride, stannous fluoride or potassium fluoride), or an antibiotic agent.

In some embodiments, a second agent can be included in the system of invention. The second agent can be same as or different from the physiologically active agent, as described above. In some embodiments, the second agent is flavoring agent, a hormone, or a fragrance.

### Controlled Release

In some embodiments of the invention system, it provides controlled delivery of a physiologically active agent into a subject in need thereof. Controlled release is achieved by the design of a delivery module disclosed herein, which is described above, including using of a carrier material, which is described in more detail below. As used herein, the term “controlled release” is used interchangeably with the term “controlled delivery.”

Various release profile of the physiologically active agent can be achieved using different configuration of the delivery module described herein. The nature of the various carrier materials, e.g., hydrophilicity, hydrophobicity, rate of degradation, etc. can be made use to achieve control of delivery to a specific target site of condition or disorder at a desired rate of release of the physiologically active agent.

In some embodiments, release of the physiologically active agent can occur over the course from about 1 hour to about one month, for example, from 1 day to about 30 days, from 1 day to about 4 weeks, from 1 day to about 3 weeks, from 1 day to about 2 weeks, from 1 day to about 1 week, from 2 days to about 30 days, from 2 days to about 4 weeks, from 2 days to about 3 weeks, from 2 days to about 2 weeks, from 2 days to about 1 week, from 3 days to about 30 days, from 3 days to about 4 weeks, from 3 days to about 3 weeks, from 3 days to about 2 weeks, from 3 days to about 1 week, from 4 days to about 30 days, from 4 days to about 4 weeks, from 4 days to about 3 weeks, from 4 days to about 2 weeks, from 4 days to about 1 week, from 5 days to about 30 days, from 5 days to about 4 weeks, from 5 days to about 3 weeks, from 5 days to about 2 weeks, from 5 days to about 1 week, from 1 day to 6 days, from 1 day to 5 days, from 1 day to 4 days, from 1 day to 3 days, from 1 day to 2 days, from 2 days to 6 days, from 2 days to 5 days, from 2 days to 4 days, from 2 days to 3 days, from 3 days to 6 days, from 3 days to 5 days, from 3 days to 4 days, from 4 days to 6 days, from 4 days to 5 days, or from 5 days to 6 days.

### Carrier Material

The carrier material is generally a material that provides controlled release of the physiologically active agent. The carrier material useful in the present invention can be any biocompatible polymer, which can be natural polymer or synthetic polymer.

In some embodiments, the carrier is a synthetic polymer, for example, a polyester.

In some embodiments, the carrier material can include any of the biodegradable micro and nanoparticles, biodegradable block copolymer micelles, biodegradable targeting micro and nanoparticles with functional polymers, responsive polymers, PEG conjugation (PEGylation), polyoxazoline conjugation, dendrimers, and polymeric hydrogels for localized delivery.

Carrier materials can be used to allow a physiologically active agent to be delivered to the right place, at the right time and in the right amount. Low drug solubility, drug degradation, drug toxicity, or rapid clearance from the body can reduce the effectiveness of an otherwise promising candidate. Synthetic and natural polymers are both effective solutions for the delivery of small molecules, proteins, genes, or peptides.

Different polymer types can be used to control drug release rates, deliver a drug to the desired site of action, or increase drug solubility and reduce toxicity (increase biocompatibility). Examples of such polymers include: Poly(L-lactide) (PLLA), poly(D,L-lactide) (PDLLA); poly(lactide-glycolide) (PLGA) of various lactide:glycolide ratio, chitosan, alginic acid sodium salt.

In some embodiments, the carrier material is a biodegradable polymer. Examples of biodegradable polymers include polylactide and polyglycolide (PLA, PGA, PLGA), RESOMER®, and chitosan.

In some embodiments, the carrier material can include a hydrophilic polymer. Examples of hydrophilic polymers are PEGs, poly(2-oxazoline), PNIPAM.

In some embodiments, the carrier material can include conjugated nanomaterials, such as Au, Ag, iron oxide nanoparticles, graphene oxide, and carbon nanotubes.

In some embodiments, the carrier material can include amphiphilic diblock copolymers consisting of a hydrophilic block (PEG) and a hydrophobic block (PLA, PGA, PLGA, PCL), which have the capacity to form micelles to encapsulate hydrophobic drugs in order to increase drug solubility and facilitate delivery. The phase segregation of polymer blocks into a micellar morphology creates an environment where hydrophobic molecules can dissolve in the hydrophobic core.

In some embodiments, the carrier material can be a film forming material

A broad range of selection of drug delivery carrier materials are commercially developed. Examples of such materials are commercially available from, for example Sigma-Aldrich.

In some embodiments, the carrier material is a material for forming the appliances or trays disclosed herein, which is described in more detail below.

### Decorative Element

As used herein, the term “decorative element” refers to an element that is aesthetic appealing or matter of value of subjectivity to a user of the invention system disclosed herein.

A decorative element useful in the present invention can be any aesthetic features that formed of a biocompatible material. For example, a decorative element can comprise a glittering material, a matter of color, such as an organic or inorganic dye or an object of physical or chemical instrumentation, a pattern, or design.

In some embodiments, the decorative element comprises a glittering material. Examples of glittering materials can be or can comprise, for example, organic crystals or inorganic crystals. In some further embodiments, a glittering material can comprise gold crystals, silver crystals, or diamond crystals.

In some embodiments, the decorative element can comprises a figurative element, a numeric element or an alphabetic element. Examples of such figurative element can be a shape of a start, a shape of heart, a human figure, an animal figure, a plant, a flower, a science or technology symbol, formula, or structure, an artistic work, a work of engineering, a natural scene, a pattern, or a design. In some embodiments, such figurative element can be of artistic work, e.g., drawing, painting, or sculpture, architecture, etc. In some further embodiments, such figurative element can be of work of engineering, e.g., a building or bridge. In some further embodiments, such figurative element can be of a natural scene, e.g., ocean, mountain, river, lake, tree, tree, forest, beach, or park.

In some embodiments, the decorative element can comprises a fragrance. Such fragrance can provide physiologically appealing senses, and, the fragrance can be, for example, senses of a scent, a sexual hormone, and/or a mouth-odor masking agent.

### Non-Transparent Surface

The non-transparent surface useful in the present invention can be formed of any biocompatible material(s). In some embodiments, the non-transparent surface can comprise microparticles. Such microparticles can comprise an inorganic oxide or an inorganic salt, for example. In some embodiments, the microparticles can comprise a material that is an oxide, a phosphate, a sulfate, a fluoride, chloride, a flavoring agent, a silicate, an aluminate, or a combination thereof.

In some further embodiments, the microparticles can comprise a material such as TiO₂, CaCO₃, Al₂O₃, SiO₂, a silicate, a sulfate, a phosphate, a fluoride, a chloride, a flavoring agent, or a combination thereof.

### Appliance-Forming Materials

The appliances can be formed of a plastic material such as a polyester. An example of such polyester is poly(lactic acid) (PLA) or a related material. Many types of polyester materials are developed for forming an appliance. Examples of such materials are also described in U.S. Pat. Nos. 5,605,459; 5,533,895; 5,474,448; 5,454,717; 5,447,432; 5,431,562; 5,395,238; 5,368,478; 5,139,419; 5,338,198; 5,452,219; 5,607,305; 5,587,912; 5,342,202; 5,340,309; 5,549,476; 5,382,164; 5,273,429; 4,936,862; 3,860,803; 3,660,900; 5,645,421; 5,055,039; 4,798,534; 4,856,991; 5,035,613; 5,059,118; 5,186,623; 4,755,139; 6,398,548 and 6,554,611. For concise of description, the teachings of appliance forming materials described in these patents are incorporated herein by reference in their entirety.

In some embodiments, the appliance forming material can also include an elastic material that may include one or more polymers. The polymer is preferably inert and biocompatible. The polymer is also sufficiently flexible to allow easy removal and application for the user, but also sufficiently rigid to allow controlled teeth movement. Any conventional material normally used in dental treatments for fabricating removable appliances can be used in this system. Specific examples of useful polymers include any elastic polymeric materials, such as those commonly used in the art of dentistry, e.g, olefin polymers or copolymers, such as polyethlyene, polybutylene, polyisobutylene, polypropylene, ethylene vinyl acetate, polyvinyl alcohol, polystyrene, copolymers that include two or more of ethylene, propylene, butylene, isobutene, pentene, styene, vinyl acetate, vinyl alcohol and a combination thereof, or a mixture thereof. The polymeric material may further include a material to modify the biocompatibility. Such biocompatibility modifying materials include, e.g., polyethylene glycol, polypropylene glycol, polyethylene oxide or a natural polymer such as cellulose or alginate, collagen, and the like.

In some embodiment, the polymeric materials may further include a reinforcing material such as fibers, chips, wires, glass fibers, carbon fibers, pieces, strips, mesh, lattices, and networks and interpenetrating networks. Some representative reinforcing materials include, for example, micro or nano aluminum oxide phases, carbon fibers, etc., or mixtures thereof.

### Method of Fabrication

In another aspect of the present invention, it is provided a method for fabricating a system for delivery of a physiologically active agent to a user in need thereof, which method comprising:

providing one or two appliances that correspond to an upper arch and/or lower dental arch of the user, each appliance comprising a labial/buccal side surface and a palatal/lingual side surface,

providing a delivery module of a physiologically active agent, comprising the physiologically active agent, and

forming the system,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the system which system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the delivery module further comprises a carrier material, and the physiologically active agent is admixed with, embedded within or encapsulated within the carrier material.

In some embodiments, optionally in combination with any one of the embodiments of invention method disclosed herein, the delivery module is embedded within the body matrix of the polymeric material or is admixed with the polymeric material forming the one or two appliances.

In some embodiments, optionally in combination with any one of the embodiments of invention method disclosed herein, the delivery module is formed of the physiologically active agent per se.

In some embodiments, optionally in combination with any one of the embodiments of invention method disclosed herein, the delivery module is a coating on a surface of the one or two appliances.

In some embodiments, optionally in combination with any one of the embodiments of invention method disclosed herein, the one or two appliances comprises a structure selected from the group consisting of micropores, microchannels, depots, and a combination thereof.

In some embodiments, optionally in combination with any one of the embodiments of invention method disclosed herein, the structure comprises discrete or interconnecting micropores, microchannels, depots, or a combination thereof.

In some embodiments, optionally in combination with any one of the embodiments of invention method disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention method disclosed herein, the physiologically active agent is insulin, a fluoride, or an antibiotic agent.

In some embodiments, optionally in combination with any one of the embodiments of invention method disclosed herein, the system further comprises a second agent, wherein the second agent is flavoring agent or a fragrance.

In some embodiments, optionally in combination with any one of the embodiments of invention method disclosed herein, the system further comprises a top layer of coating on a surface of the one or two appliances, wherein the top layer comprises the second agent and an optional carrier material.

In some embodiments, optionally in combination with any one of the embodiments of invention method disclosed herein, the second agent is a female or male sex hormone.

In some embodiments, optionally in combination with any one of the embodiments of invention method disclosed herein, the second agent is a flavoring agent selected from a taste of chocolate, vanilla, mint, a fruit, or candy.

In some embodiments, optionally in combination with any one of the embodiments of invention method disclosed herein, the second agent is a fragrance capable of masking a bad breath or mouse odor.

In some embodiments, optionally in combination with any one of the embodiments of invention method disclosed herein, the one or more appliances further comprise a decorative surface.

In some embodiments, optionally in combination with any one of the embodiments of invention method disclosed herein, the one or more appliances are orthodontic aligning appliances.

In some embodiments, optionally in combination with any one of the embodiments of invention method disclosed herein, the occlusal bottoms of at least one or more of the polymeric shells are removed such that the occlusal surfaces of at least one or more of the polymeric shells are exposed

The various embodiments of the delivery module can be readily fabricated according to established methods. For example, a biodegradable polymer can be used as a matrix material to mix with a physiologically active agent. The mixture, as a delivery module of the physiologically agent, has a rate of release of the agent at the rate of degradation of the polymer so as to provide control of release of the physiologically active agent. The mixture can be incorporated into a polymeric material forming the appliances of the present invention or coated onto a surface of the appliance

In some embodiments, a physiologically active agent can be incorporated directly, without a carrier material, to a polymeric material forming the appliance of the invention without a carrier. In some embodiments, the agent can be coated directly without a carrier material on a surface of the appliance of invention.

In some embodiments, a physiologically active agent can form microparticles or nanoparticles with or without a carrier material. Such microparticles or nanoparticles can be incorporated into a polymeric material forming the appliances of the present invention or coated onto a surface of the appliance.

In some embodiments, a physiologically active agent can be encapsulated within a carrier materials, forming microcapsules or nanocapsules. Such microcapsules or nanocapsules can be incorporated into a polymeric material forming the appliances of the present invention or coated onto a surface of the appliance.

Incorporation of the various embodiments of delivery module into the appliance of invention can be achieved by simple mixture with a polymeric material forming the appliance of invention. Alternatively, the various embodiments of delivery module into the appliance of invention can be achieved by coating the delivery module to a surface of the appliance of invention. As a further option, the delivery module of the various embodiments can be loaded to an appliance by a) forming pores, channels, or depots on the appliance, and loading the delivery module of the various embodiments to the pores, channels, or depots on the appliance.

Methods of forming a coating on a surface of appliance can be, for example, dip-coating, spray coating using a suspension or solution of the physiologically active agent, etc. Such methods are well documented in the art.

Methods of forming micropores, microchannels, or microdepots are be readily achieved by, e.g., laser drilling, solvent erosion, heat carving, mechanical drilling, for example. Loading of the physiologically active agent and/or a delivery module of various embodiments can be achieved by dip-loading or soaking a suspension or solution of the physiologically active agent or the delivery module. Such methods are well documented in the art.

### Method of Forming Appliances

The appliance, as an orthodontic aligner, can be formed by (a) receiving prescribed orthodontic information for a user in need of orthodontic treatment, (b) forming a cycle of appliances comprising individual appliances, and (c) forming a cycle of appliances.

Generally, the appliances can be formed by (1) generating/obtaining an initial data set such as an initial digital data set (IDDS) representing the initial tooth arrangement, (2) generating a digital data set (DDS) or non-digital data set (NDDS) representing an intermediate tooth arrangement, (3) generating an end or a final data set such as a DDS or NDDS representing an end tooth arrangement or a final tooth arrangement, and (4) optionally producing a plurality of successive digital data sets based on both of the first digital data set and the final digital data set, wherein the plurality of successive data sets represent a series of successive tooth arrangements progressing from the intermediate tooth arrangement last end tooth arrangement to the end tooth arrangement or the final tooth arrangement, and (5) forming an appliance or a plurality of appliances based on the digital data sets. In some embodiments, the digital data sets can be converted into visual images representing a tooth arrangement, and the appliances can be formed based on the visual images. Methods of obtaining the IDDS and DDS, generating a visual image based on DDS and forming an appliance based on the visual image are described in U.S. Pat. Nos. 6,398,548; 6,544,611; 5,895,893; 6,244,861; 6,616,444; 5,645,420; and 5,447,432, the teachings of which are incorporated herein by reference.

The initial digital data set may be provided by any techniques known in the art, including digitizing X-ray images, images produced by computer-aided tomography (CAT scans), images produced by magnetic resonance imaging (MRI), images produced by photo scanning, and the like. The images will be three-dimensional images and digitization may be accomplished using known technology. For example, the initial digital data set is provided by producing a plaster cast of the user's teeth (prior to treatment) by techniques known in the art. The plaster cast so produced may then be scanned using laser or other scanning equipment to produce a high resolution digital representation of the plaster cast of the user's teeth.

In a preferred embodiment, a wax bite is also obtained from the user using standard methods. The wax bite allows plaster casts of a user's upper and lower dentition to be placed relative to one another in the centric occlusal position. The pair of casts then can be scanned to provide information on the relative position of the jaw in this position. This information is then incorporated into the IDDS for both arches.

Once the digital data set is acquired, an image can be presented and manipulated on a suitable computer system equipped with computer-aided design software, as described in greater detail below. The image manipulation will usually comprise defining boundaries about at least some of the individual teeth, and causing the images of the teeth to be moved relative to the jaw and other teeth by manipulation of the image via the computer. Methods are also provided for detecting cusp information for the teeth. The image manipulation can be done entirely subjectively, i.e. the user may simply reposition teeth in an aesthetically and/or therapeutically desired manner based on observation of the image alone. Alternatively, the computer system could be provided with rules and algorithms which assist the user in repositioning the teeth. In some instances, it will be possible to provide rules and algorithms which reposition the teeth in a fully automatic manner, i.e. without user intervention. Once the individual teeth have been repositioned, a final digital data set representing the desired final tooth arrangement will be generated and stored.

An exemplary method for determining the final tooth arrangement is for the treating professional to define the final tooth positions, e.g. by writing a prescription. The use of prescriptions for defining the desired outcomes of orthodontic procedures is well known in the art. When a prescription or other final designation is provided, the image can then be manipulated to match the prescription. In some cases, it would be possible to provide software which could interpret the prescription in order to generate the final image and thus the digital data set representing the final tooth arrangement.

In yet another aspect, methods described herein are provided for producing a plurality of digital data sets representing a series of discrete tooth arrangements progressing from an initial tooth arrangement to a final tooth arrangement. Such methods comprise providing a digital data set representing an initial tooth arrangement (which may be accomplished according to any of the techniques set forth above). A digital data set representing a final tooth arrangement is also provided. Such final digital data set may be determined by the methods described previously. A plurality of successive digital or non-digital data sets are then produced based on the initial digital data set and the final digital data set. Usually, the successive digital data sets are produced by determining positional differences between selected individual teeth in the initial data set and in the final data set and interpolating said differences. Such interpolation may be performed over as many discrete stages as may be desired, usually at least three, often at least four, more often at least ten, sometimes at least twenty-five, and occasionally forty or more. Many times, the interpolation will be linear interpolation for some or all of the positional differences. Alternatively, the interpolation may be non-linear. In a preferred embodiment, non-linear interpolation is computed automatically by the computer using path scheduling and collision detection techniques to avoid interferences between individual teeth. The positional differences will correspond to tooth movements where the maximum linear movement of any point on a tooth is 2 mm or less, usually being 1 mm or less, and often being 0.5 mm or less.

Often, the user will specify certain target intermediate tooth arrangements, referred to as “key frames,” which are incorporated directly into the intermediate digital data sets. The methods of the present system then determine successive digital data sets between the key frames in the manner described above, e.g. by linear or non-linear interpolation between the key frames. The key frames may be determined by a user, e.g. the individual manipulating a visual image at the computer used for generating the digital data sets, or alternatively may be provided by the treating professional as a prescription in the same manner as the prescription for the final tooth arrangement.

In still another aspect, methods described herein provide for fabricating a plurality of dental incremental position adjustment appliances. Said methods comprise providing an initial digital data set, a final digital or non-digital data set, and producing a plurality of successive digital or non-digital data sets representing the target successive tooth arrangements, generally as just described. The dental appliances are then fabricated based on at least some of the digital data sets representing the successive tooth arrangements. Preferably, the fabricating step comprises controlling a fabrication machine based on the successive digital data sets to produce successive positive models of the desired tooth arrangements. The dental appliances are then produced as negatives of the positive models using conventional positive pressure or vacuum fabrication techniques. The fabrication machine may comprise a stereolithography or other similar machine which relies on selectively hardening a volume of non-hardened polymeric resin by scanning a laser to selectively harden the resin in a shape based on the digital data set. Other fabrication machines which could be utilized in the methods of the present system include tooling machines and wax deposition machines.

In still another aspect, methods of the present system for fabricating a dental appliance comprise providing a digital data set representing a modified tooth arrangement for a user. A fabrication machine is then used to produce a positive model of the modified tooth arrangement based on the digital data set. The dental appliance is then produced as a negative of the positive model. The fabrication machine may be a stereolithography or other machine as described above, and the positive model is produced by conventional pressure or vacuum molding techniques.

In a still further aspect, methods for fabricating a dental appliance described herein comprise providing a first digital data set representing a modified tooth arrangement for a user. A second digital data set is then produced from the first digital data set, where the second data set represents a negative model of the modified tooth arrangement. The fabrication machine is then controlled based on the second digital data set to produce the dental appliance. The fabrication machine will usually rely on selectively hardening a non-hardened resin to produce the appliance. The appliance typically comprises a polymeric shell having a cavity shape to receive and resiliently reposition teeth from an initial tooth arrangement to the modified tooth arrangement.

In some embodiments, the orthodontic doctor can take an imprint or scan a last intermediate tooth arrangement after the user has undergone the treatment of one or more cycles of appliances. A digital data set of the last intermediate tooth arrangement of the previous cycle thus can be obtained based on the imprint or scan. This digital data set of the last intermediate tooth arrangement of the previous cycle is then used as the initial point for generating a new set of digital data and visual images based on the new set of digital data representing one or more new intermediate tooth arrangements and a final tooth arrangement for the fabrication of a new cycle of appliances.

In some embodiments, the final tooth arrangement can be achieved with the application of two or more cycles of appliances, and each cycle of the appliances incrementally move the teeth starting from the tooth arrangement positioned by the last appliance of the previous cycle. Cycles of appliances can therefore be made according to the principles described above.

### Method of Use

In a further aspect of the present invention, it is provided a method of treating or ameliorating a condition in a user, comprising delivering to a user in need thereof a physiologically active agent to the user using a system, the system comprising:

one or two appliances that correspond to an upper arch and/or lower dental arch of the user, each appliance comprising a labial/buccal side surface and a palatal/lingual side surface,

a delivery module of a physiologically active agent, comprising the physiologically active agent,

wherein the one or two appliances comprise a body matrix of a polymeric material comprising polymeric shells, each shell having the geometry corresponding to one tooth of the dental arch(s) and configured to receive the one tooth, and

wherein the delivery module provides a controlled release of the physiologically active agent.

In some embodiments, optionally in combination with any one of the embodiments of invention method disclosed herein, the delivery module further comprises a carrier material, and the physiologically active agent is admixed with, embedded within or encapsulated within the carrier material.

In some embodiments, optionally in combination with any one of the embodiments of invention method disclosed herein, the delivery module is embedded within the body matrix of the polymeric material or is admixed with the polymeric material forming the one or two appliances.

In some embodiments, optionally in combination with any one of the embodiments of invention method disclosed herein, the delivery module is formed of the physiologically active agent per se.

In some embodiments, optionally in combination with any one of the embodiments of invention method disclosed herein, the delivery module is a coating on a surface of the one or two appliances.

In some embodiments, optionally in combination with any one of the embodiments of invention method disclosed herein, the one or two appliances comprises a structure selected from the group consisting of micropores, microchannels, depots, and a combination thereof.

In some embodiments, optionally in combination with any one of the embodiments of invention method disclosed herein, the structure comprises discrete or interconnecting micropores, microchannels, depots, or a combination thereof.

In some embodiments, optionally in combination with any one of the embodiments of invention method disclosed herein, the physiologically active agent is selected from the group consisting of a protein, a peptide, a small molecule therapeutic agent, an antimicrobial agent, and a combination thereof.

In some embodiments, optionally in combination with any one of the embodiments of invention method disclosed herein, the physiologically active agent is insulin, a fluoride, or an antibiotic agent.

In some embodiments, optionally in combination with any one of the embodiments of invention method disclosed herein, the system further comprises a second agent, wherein the second agent is flavoring agent or a fragrance.

In some embodiments, optionally in combination with any one of the embodiments of invention method disclosed herein, the system further comprises a top layer of coating on a surface of the one or two appliances, wherein the top layer comprises the second agent and an optional carrier material.

In some embodiments, optionally in combination with any one of the embodiments of invention method disclosed herein, the second agent is a female or male sex hormone.

In some embodiments, optionally in combination with any one of the embodiments of invention method disclosed herein, the second agent is a flavoring agent selected from a taste of chocolate, vanilla, mint, a fruit, or candy.

In some embodiments, optionally in combination with any one of the embodiments of invention method disclosed herein, the second agent is a fragrance capable of masking a bad breath or mouse odor.

In some embodiments, optionally in combination with any one of the embodiments of invention method disclosed herein, the one or more appliances further comprise a decorative surface.

In some embodiments, optionally in combination with any one of the embodiments of invention method disclosed herein, the one or more appliances are orthodontic aligning appliances.

In some embodiments, optionally in combination with any one of the embodiments of invention method disclosed herein, the occlusal bottoms of at least one or more of the polymeric shells are removed such that the occlusal surfaces of at least one or more of the polymeric shells are exposed.

In some embodiments, optionally in combination with any one of the embodiments of invention method disclosed herein, the condition is a chronic condition or an acute-on-chronic condition.

In some embodiments, optionally in combination with any one of the embodiments of invention method disclosed herein, the condition is a dental condition.

In some embodiments, optionally in combination with any one of the embodiments of invention method disclosed herein, the condition is a periodontal condition.

In some embodiments, optionally in combination with any one of the embodiments of invention method disclosed herein, the condition is diabetic condition or a neurodegernative condition.

While the above is a complete description of the preferred embodiments of the system, various alternatives, modifications, and equivalents may be used. Therefore, the above description should not be taken as limiting the scope of the system which is defined by the appended claims.

I claim:

1. A system for delivery of a physiologically active agent to a user in need thereof, comprising:
one or two appliances that correspond to an upper arch and/or lower dental arch of the user, each appliance comprising a labial/buccal side surface and a palatal/lingual side surface,
a delivery module of a physiologically active agent, comprising the physiologically active agent,
wherein the one or two appliances comprise a body matrix of a polymeric material comprising polymeric shells, each shell having the geometry corresponding to one tooth of the dental arch(s) and configured to receive the one tooth, and
wherein the delivery module provides a controlled release of the physiologically active agent.

2. The system according to claim 1, wherein the delivery module further comprises a carrier material, and
wherein the physiologically active agent is admixed with, embedded within or encapsulated within the carrier material.

3. The system according to claim 1, wherein the delivery module is embedded within the body matrix of the polymeric material or is admixed with the polymeric material forming the one or two appliances.

4. The system according to claim 3, wherein the delivery module is formed of the physiologically active agent per se.

5. The system according to claim 1, wherein the delivery module is a coating on a surface of the one or two appliances.

6. The system according to claim 1, wherein the one or two appliances comprises a structure selected from the group consisting of micropores, microchannels, depots, and a combination thereof.

7. The system according to claim 6, wherein the structure comprises discrete or interconnecting micropores, microchannels, depots, or a combination thereof.

8. The system according to claim 1, wherein the physiologically active agent is selected from the group consisting of a protein, a peptide, a small molecule therapeutic agent, an antimicrobial agent, and a combination thereof.

9. The system according to claim 1, wherein the physiologically active agent is insulin, a fluoride, or an antibiotic agent.

10. The system according to claim 1, further comprising a second agent, wherein the second agent is flavoring agent or a fragrance.

11. The system according to claim 10, further comprising a top layer of coating on a surface of the one or two appliances, wherein the top layer comprises the second agent and an optional carrier material.

12. The system according to claim 11, wherein the second agent is a female or male sex hormone.

13. The system according to claim 12, wherein the second agent is a flavoring agent selected from a taste of chocolate, vanilla, mint, a fruit, or candy.

14. The system according to claim 11, wherein the second agent is a fragrance capable of masking a bad breath or mouse odor.

15. The system according to claim 1, wherein the one or more appliances are orthodontic aligning appliances.

16. A method for fabricating a system for delivery of a physiologically active agent to a user in need thereof, comprising:
providing one or two appliances that correspond to an upper arch and/or lower dental arch of the user, each appliance comprising a labial/buccal side surface and a palatal/lingual side surface,
providing a delivery module of a physiologically active agent, comprising the physiologically active agent, and
forming the system,
wherein the one or two appliances comprise a body matrix of a polymeric material comprising polymeric shells, each shell having the geometry corresponding to one tooth of the dental arch(s) and configured to receive the one tooth, and
wherein the delivery module provides a controlled release of the physiologically active agent.

17. A method of treating or ameliorating a condition in a user, comprising delivering to a user in need thereof a physiologically active agent to the user using a system, the system comprising:
one or two appliances that correspond to an upper arch and/or lower dental arch of the user, each appliance comprising a labial/buccal side surface and a palatal/lingual side surface,
a delivery module of a physiologically active agent, comprising the physiologically active agent,
wherein the one or two appliances comprise a body matrix of a polymeric material comprising polymeric shells, each shell having the geometry corresponding to one tooth of the dental arch(s) and configured to receive the one tooth, and
wherein the delivery module provides a controlled release of the physiologically active agent.

18. The method according to claim 17, wherein the condition is a chronic condition or an acute-on-chronic condition.

19. The method according to claim 17, wherein the condition is a dental condition, a periodontal condition, a diabetic condition, or a neurodegernative condition.
